(19) United States
(12) Patent Application Publication
(10) Pub. No.: US 2007/0156154 A1
(43) Pub. Date: Jul. 5, 2007

(54) Locking Screw

(76) Inventors: Andre Schlienger, Basel (CH); Peter Senn, Waldenburg (CH); Markus Buettler, Oensingen (CH)

Correspondence Address: JONES DAY, 222 EAST 41ST STREET, NEW YORK, NY, 10017-6702, US

(21) Appl. No.: 11/625,893

(22) Filed: Jan. 23, 2007

Related U.S. Application Data
(63) Continuation of application No. PCT/CH04/00469, filed on Jul. 23, 2004.
(63) Continuation-in-part of application No. 11/237,595, filed on Sep. 27, 2005.
Application No. 11/237,595 is a continuation of application No. PCT/CH03/00202, filed on Mar. 28, 2003.

(51) Int. Cl. A61B 17/58

(52) U.S. Cl. 606/73

(57) ABSTRACT

A screw comprising a head-side end, a tip, a screw core comprising an envelope surface carrying an outer thread, and a central line formed by the connection line of the centres of gravity of the axially successive, orthogonal cross-sectional surfaces of the screw core, without taking into account any cavities. The screw core comprises at least one cavity and the central line is not straight. The material weakening of the screw core, caused by the cavities, simplifies the passage thereof through a narrow point e.g. in a transverse borehole of an intrameduliar nail, as the screw without a straight central line can be radially deformed to a certain degree.

### CROSS-REFERENCE TO RELATED APPLICATIONS

This application is a continuation of International Application No. PCT/CH2004/000469, filed Jul. 23, 2004. This application is also a continuation-in-part of U.S. application Ser. No. 11/237,595, filed Sep. 27, 2005, which is a continuation of International Application No. PCT/CH2003/000202, filed Mar. 28, 2003. Each of the above references is expressly incorporated by reference herein.

### FIELD OF THE INVENTION

The invention relates to a locking screw for an intramedullary nail with transverse boreholes.

### BACKGROUND OF THE INVENTION

The locking of intramedullary nails is prior art. Locking screws are introduced into the transverse boreholes of an intramedullary either with the help of an imaging process (x-ray control) or of a more or less complicated targeting device. In either case, a certain targeting accuracy is unavoidable, that is, the tip of the screw cannot be aligned precisely coaxially with the center axis of the transverse borehole and, instead, deviates from this by a certain amount. So that the locking screw ends up in the transverse borehole and can be brought through the latter in spite of this targeting error, the external diameter of the screw is underdimensioned relative to the diameter of the transverse borehole. If this targeting inaccuracy remains within the boundaries of this underdimensioning, the locking screw can be guided through the transverse borehole without problems in despite of the targeting error. However, because of the underdimensioning, the locking screw now has a certain clearance relative to the transverse borehole,

This clearance defines the amount, by which the main bone fragments, which are to be fixed by means of locking screws in the corresponding locking hole, can move relative to the nail and, accordingly, because of the rigidity of the nail, also relative to the other main bone fragments, which are to be fastened with the same nail. Together with the flexibility of the material and of the overall device, this may cumulatively assume a magnitude, which prevents or substantially delays successful healing. Admittedly, in order to guarantee the applicability of the locking for the surgeon, this clearance is unavoidable. Clinically, however, it is undesirable for certain indications, such as in the case of metaphysical fragments.

Even nails with a solid cross section, which may have internal thread in the locking hole, are not free of clearance. The internal thread only prevents axial movement of the nail on the locking screw.

### SUMMARY OF THE INVENTION

The invention is to provide a remedy here. It is an object of the invention to provide a locking screw, with which the existing clearance between it and the transverse borehole in a locking intramedullary nail can be eliminated and a non-positive tension between the screw and the intramedullary nail can be produced

The concept of the central line is defined by the line connecting the centers of gravity of the axially successive orthogonal cross-sectional areas of the core of the screw without taking into consideration any cavities and disregarding the external thread.

The advantages, achieved by the invention, may be seen to lie essentially therein that, due to the inventive locking screw, the clearance between the transverse borehole of the intramedullary nail and the locking screw can be eliminated. The following are further advantages:

the insertion accuracy and the time expended by the surgeon remain within the previous limits;

the strength of the locking screw is retained;

should it break, the screw can be extracted;

the weakening of the screw core, brought about by the cavities, facilitates the advance of the latter through a constricted site, such as a transverse borehole, of an intramedullary nail, because the screws, which do not have a straight, central line, can be deformed radially to a certain degree.

For a special embodiment of the invention, the screw core has a cavity in the form of an axial canalization, which passes preferably continuously through the screw, so that a guiding wire may also be used.

The central line of the screw may extend helically or spirally, resulting in an axial displacement of the screw, which leads to a gradual elimination of the clearance and a gradual build-up of the tension

The central line may lie in a single plane, in which case it is particularly easy to manufacture the screw.

Preferably, a marking is provided at the head end of the screw. Advantageously, this marking shows the orientation of the plane in which any points of inflection of the central line are located.

The central line may also not lie in one plane. By these means, it becomes easier for the screw to wind its way through the borehole and the stress points do not lie on one line.

The central line may be formed, for example, of several straight lines, which are misaligned with respect to one another.

In the case of a special embodiment, the circumferential surface of the screw core is not formed from a family of parallel straight lines, Accordingly, the circumferential surface correspond not to the circumferential surface of a straight circular cylinder but to that of a circular cylinder, which is deformed according to the central line, which is not rectilinear.

The volume V_{hollow} of the screw core having one or more cavities advantageously amounts to less than 95% of the volume V_{full} of the body surrounded by the circumferential surface of the screw core. Typically, V_{hollow}<0.90 V_{full} and preferably V_{hollow}<0.80 V_{full}.

With the exception of any axial exit of a cavity at the tip of the screw, the cavities in the screw core should penetrate advantageously not more than 1% and preferably 0% of the circumferential surface.

The tip of the screw may taper and the head end may have means for driving the screw.

In the case of a special embodiment, the screw core does not have a rotational axis of symmetry.

For a further embodiment, a straight line, extending between the center of the head end and the center of the tip, is at a distance x>0 from the central line at least in placers. Advantageously, the distance “x” fulfills the condition that 0.02 d<x<0.6 d and preferably the condition that 0.1 d<x<0.4 d, “d” corresponding to the diameter of the external thread. At least on a partial axial segment, the distance “x” should advantageously be greater than 0.05 mm and preferably greater than 0.50 mm.

For a further embodiment, the central line, extending between the head end and the tip, has a length L and is divided into three sections A, B and C,
A) section A extending from the head end (5) by the amount of 0.10 L to 0.25 L in the direction of the tip,
B) section B extending from the tip the amount of 0.10 to 0.25 L in the direction of the head end,
C) section C being disposed between the sections A and B and having the length C=(L−A−B) and
D) the central line in the sections A and B extending essentially in a straight line and being co-axial with one another.

This results in the advantage that the locking takes place in the opposite corticalis by the rotational movement about the central line and that the locking in the corticalis at the head end takes place essentially with the proper axial alignment.

In the case of a further embodiment, the central line is curved S-shaped or eccentric preferably only in section C. Moreover, preferably it has a point of inflection only in section C. In the case of a further embodiment, the central line has at least two points of inflection at a distance of y from one another preferably only in section C.

in the case of the special embodiment, the wall thickness W of the screw core in the region of the cannulation is larger than 0.05 d and preferably larger than 0.1 d. Advantageously, the wall thickness W of the screw core in the region of the cannulation is less than 0.8 d and preferably less than 0.6 d.

The inventive screw can be used together with a looking intramedululary nail, which has at least one transverse borehole, which has a cross-sectional profile P with a maximal extent “a” measured in the direction of the central line and a maximum extent b measured perpendicular to “a”, with the proviso that, on the one hand, a>b and, on the other, a>d<b. The cross-sectional profile P, however, may also be circular with a=b. Advantageously, the condition that 0.70 b<d<0.95 b and preferably 0.8 b<d<0.9 is fulfilled.

The distance x advantageously fulfills the condition that x<(b−d+1 mm), “b” being the diameter of the transverse borehole in mm and “d” the diameter of the external thread in mm.

The distance x advantageously should fulfill the condition that 0.5(b−d)<x<0.35(b−d) and preferably the condition that 1.5(b−d)<x<2.2(b−d), “d” being the diameter of the external thread in mm.

The diameter D should fulfill the condition that 0.02 d<D<0.60 d and preferably the condition that 0.1 d<x<0.4 d:, “d” being the diameter of the external thread.

The distance y between two adjacent points of inflection should essentially fulfill the condition that D=ny, n being an odd number and D the diameter of the intramedullary nail.

### BRIEF DESCRIPTION OF THE DRAWINGS

The invention and further developments of the invention are explained in even greater detail in the following by means of partially diagrammatic representations of several examples, of which

FIG. 1 shows a perspective view of an inventive screw,

FIG. 2 shows a longitudinal section through the screw of FIG. 1 and

FIG. 3 shows a longitudinal section through a locking intramedullary nail with a transverse borehole, into which the screw of FIG. 1 has been introduced.

### DETAILED DESCRIPTION OF THE INVENTION

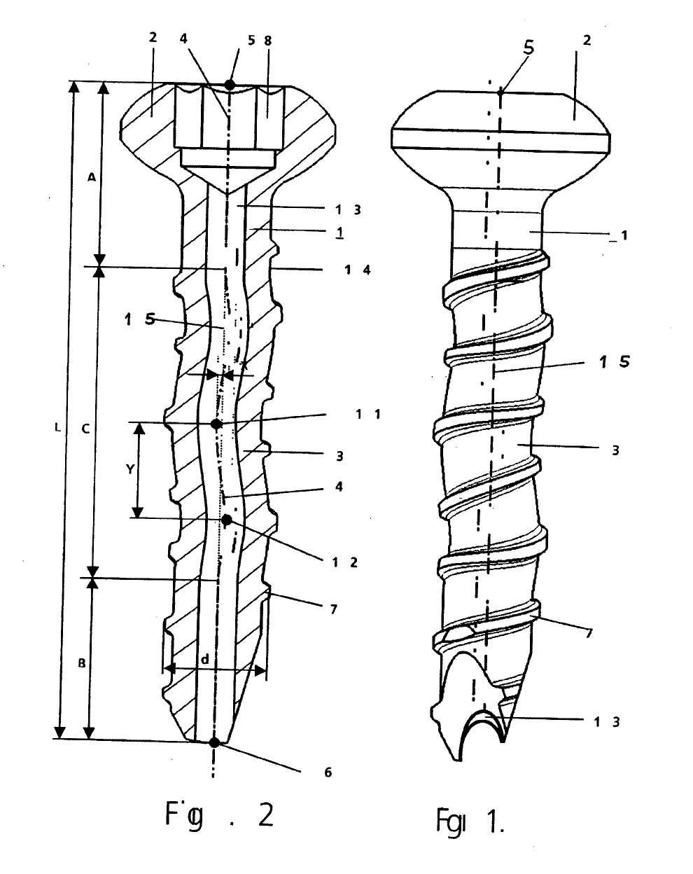

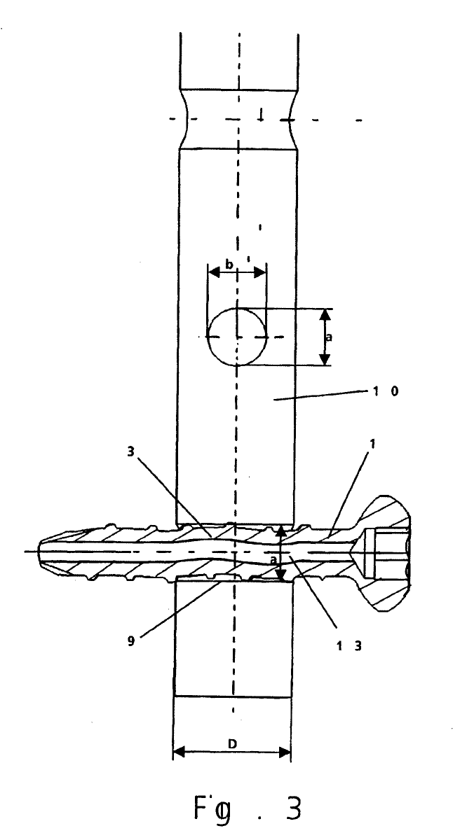

The screw, shown in FIGS. 1 and 2, comprises a screw head 2 with a hexagon socket 8, a screw core 3 with an external thread 7 and a central line 4, which extends from the head end 5 to the tip 6 of the screw. The central line 4 is not a straight line, as it is in the case of the usual straight screws. Instead, in the example shown, in the length region C, which is defined below, it consists of a serpentine line, which lies in the plane of the drawing and has two points of inflection 11, 12, The straight line 15, passing through the center of the head end 5 and of the tip 6, therefore deviates in places from the central line 4 by the variable amount x. In the example shown, the maximum deviation of x, measured at the points of inflection of the central line 4, is 0.2 mm.

Between the center of the head end 5 and that of the tip 6, which are at a distance L from one another, the central line 4 is divided into three sections A, B and C. Section A extends from the center 5 at the screw head 2 by the amount of ⅙ L in the direction of the tip 6 at the free end of the screw core 3 in essentially a straight line. Section B extends from the tip 6 at the free end of the screw core 3 by the amount of ⅙ L in the direction of the center of the end 5 of the screw head 2 and also extends essentially linearly as well as coaxially with section A. Section C is disposed between sections A and B and has the curvature described above.

Instead of being serpentine or helical, the cavity 13, in the form of a cannulation extending axially, may also be linear. Such a shape is easier to produce.

FIG. 3 shows how the screw 1 is introduced into the transverse borehole 9 of an intramedullary nail 10. The external thread 7 of the screw core 3 has a diameter “d” (FIG. 2), which is smaller than the dimension “a” of the transverse borehole 9.

The technique of screwing the locking screw into the transverse borehole of an intramedullary nail is explained briefly in the following:

the surgeon screws the screw 1 in the usual manner through the transverse borehole 9 of the intramedullary nail 10;

the relatively thin and soft corticalis yields as the screw 1 wriggles its way through so that there is no strain over the thickness of the corticalis;

in the region of the intramedullary nail 10, the screw 1 is stretched and strained somewhat because of the reaction of the wall of the transverse borehole, so that a greater force is required to screw in the screw and an increased holding force results;

the screw 1 wriggles through the inlet opening of the transverse borehole 9 of the intramedullary nail 10. Since the diameter D of the intramedullary nail 10 is larger than the distance y between the two points of inflection 11, 12, the screw 1 is braced in the transverse borehole 9. Due to the screwing-in torque or due to the force exerted by the surgeon on the screw 1, the latter is forcibly deformed elastically when it takes hold of the opposite corticalis. This leads to additional bracing of the screw 1 and to an angularly stable locking of the intramedullary nail 10.

What is claimed:

1. A screw with a head end, a tip, a screw core with one or more cavities and with circumferential surface, which carries an external thread, as well as a central line, which is formed by the line connecting the centers of gravity of the axially successive, orthogonal cross-sectional surfaces of the screw core disregarding any cavities and disregarding the external thread,
characterized in that
A) the screw core has one or more cavities and
B) the central line is not rectilinear.

2. The screw of claim 1, characterized in that the screw core has a cavity in the form of an axial cannuliation, which passes through the screw preferably continuously.

3. The screw of claim 1, characterized in that the central line proceeds helically or spirally.

4. The screw of claim 1, characterized in the central line lies in one plane.

5. The screw of claim 1, characterized in that a marking is provided at the head end of the screw.

6. The screw of claim 5, characterized in that the marking marks the orientation of the plane, in which any points of inflection of the central line are located, is marked.

7. The screw of claim 1, characterized in that the central line does not lie in one plane.

8. The screw of claim 1, characterized in that the central line is formed from several straight lines, which are misaligned with one another.

9. The screw of claim 1, characterized in that the circumferential surface is not formed from a family of parallel straight lines.

10. The screw of claim 1, characterized in that the volume V_{hollow} of the screw core, having one or more cavities, is less than 95% of the volume V_{full} of the body enclosed by the circumferential surface of the screw core.

11. The screw of claim 10, charactenzed in that V_{hollow}<0.90 V_{full}.

12. The screw of claim 1, characterized in that the cavities, excluding any axial outlet of a cavity at a tip, penetrate not more than 1% of the circumferential surface.

13. The screw of claim 1, characterized in that its tip is tapered.

14. The screw of claim 1, charactenzed in that the head end has means for driving the screw.

15. The screw of claim 1, characterized in that the screw core does not have an axis of rotational symmetry.

16. The screw of claim 1, characterized in that a straight line, extending between the center of the head end and the center of the tip, is at a distance of x>0 from the center line at least in places.

17. The screw of claim 16, characterized in that the external thread has the diameter “d” and the distance “x” fulfills the condition that 0.02 d<x<0.6 d.

18. The screw of claim 16, characterized in that the distance “x” is greater than 0.05 mm at least on a partial axial segment.
